(19) United States
(12) Patent Application Publication
(10) Pub. No.: US 2012/0259011 A1
(43) Pub. Date: Oct. 11, 2012

(54) PERSONAL CARE COMPOSITIONS WITH CATIONIC DIESTERS AND DIHYDROXYPROPYL QUATERNARY AMMONIUM SALTS

(75) Inventor: LUIS ROBERTO MISSO, Stratford, CT (US)

(73) Assignee: CONOPCO, INC., D/B/A UNILEVER, Englewood Cliffs, NJ (US)

(21) Appl. No.: 13/084,097

(22) Filed: Apr. 11, 2011

(51) Int. Cl. A61K 8/92; A61Q 19/00

(52) U.S. Cl. 514/547

(57) ABSTRACT

A personal care composition is provided which includes a diester quat and a polyhydroxy quaternary ammonium salt. Preferably the composition is a lotion or cream which exhibits a dragginess in topical application onto the skin.

### BACKGROUND OF THE INVENTION

1. Field of the Invention

The invention concerns personal care compositions formulated with cationic diesters and dihydroxypropyl quaternary ammonium salts.

2. The Related Art

Amongst some groups of consumers, personal care compositions such as creams or lotions are at least partially judged effective by their dragginess during skin rub on. Dragginess in the minds of these consumers indicates the benefit of a faster absorption.

Cationic esters have been exploited in topical cosmetics for their conditioning and pleasant skinfeel properties. U.S. Patent Application Publication No. 2008/0305056 A1 (Jenni et al.) reports the use of “ester quats” which are quaternized fatty acid alkanolamine esters and salts thereof for use in lotions and creams. Formulas with these materials exhibit low viscosity properties and have long-term storage stability. U.S. Pat. No. 5,759,557 (Epstein et al.) describes cationic emulsifiers such as dimethyl distearyl ammonium halide in combination with glycerin and a weakly acidic material providing a finished oil-in-water emulsion of pH 2.5 to 4.5. Another low pH skin treatment composition is found in U.S. Pat. No. 5,654,341 (Struewing). Therein a combination of cationic surfactant with a fatty acid is reported to form a complex preventing the cationic surfactant from irritating the skin while simultaneously substantially reducing pH of the compositions. U.S. Pat. No. 4,389,418 (Burton) describes skin care compositions that moisturize and condition based on a water-and-oil emulsion of petrolatum or mineral oil, a quaternary ammonium emulsifier, a fatty alcohol and a fatty ester emollient. The compositions are non-greasy and impart acceptable feel when applied to the skin.

Dihydroxypropyl quaternary ammonium salts (hereinafter “glycerol quats”) have been reported as exceptional skin moisturizing agents and detackifiers. Representative disclosures include U.S. Pat. No. 7,659,233 B2 (Hurley et al.), U.S. Pat. No. 7,282,471 B2 (Harichian et al.) and EP 1 809 235 B1, all to Unilever.

None of the disclosures on cationic esters or dihydroxypropyl quaternary ammonium salts have ever indicated either of these materials could impart dragginess during application onto a skin surface.

### SUMMARY OF THE INVENTION

A personal care composition is provided which includes:
(i) from about 0.05 to about 10% by weight of a diester quat of the following structure (I):

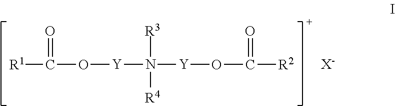

wherein R¹ and R² are independently selected from branched or unbranched, saturated or unsaturated radicals having from 13 to 23 carbon atoms; R³ and R⁴ are independently selected from an alkyl radical having from 1 to 4 carbon atoms; Y is independently selected from saturated or unsaturated alkylene radicals having from 2 to 8 carbon atoms; and X is an organic or inorganic anion;
(ii) from about 0.05% to about 10% by weight of a friction enhancing agent which is a salt of structure AB,
   wherein A is a cationic charged component of the salt AB,
   B is an anionic charged component of the salt AB, and
   A has at least one quaternized nitrogen atom, at least two hydroxyl groups and a molecular weight no higher than about 400; and
(iii) from about 1% to about 98% by weight of a cosmetically acceptable carrier.

### DETAILED DESCRIPTION OF THE INVENTION

Now it has been found that personal care compositions, particularly creams and lotions, formulated with diester quats can be rendered draggy during rub-in when topically applied to the skin. The draggy benefit has been achieved by combining the diester quat with a salt AB, the A having one quaternized nitrogen atom, at least two hydroxyl groups and a molecular weight no higher than about 400. B is an anionic charged component of the salt.

Diester quats for use in the present invention may be selected from compounds having the formula (I):

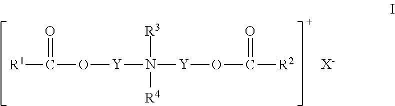

wherein R¹ and R² are independently selected from branched or unbranched, saturated or unsaturated radicals having from 13 to 23 carbon atoms; preferably having from 15 to 19 carbon atoms, particularly preferably having from 15 to 17 carbon atoms; R³ and R⁴ are independently selected from alkyl radicals having from 1 to 4 carbon atoms, preferably methyl or ethyl; Y is independently selected from saturated or unsaturated alkylene radicals having from 2 to 8 carbon atoms, preferably methylene or ethylene; and X is an organic or inorganic anion, preferably a halide, a C₁-C₄ alkyl sulfate or alkyl phosphate, and particularly preferably chloride, methosulfate or ethosulfate.

Examples of diester quats include N,N-di(β-stearoylethyl)-N,-N-dimethylammonium salt of halide or methosulfate; N,N-di(β-palmitoylethyl)-N,N-dimethylammonium salt of halide or methosulfate; N,N-di(β-oleioylethyl)-N,N-diethylammonium salt of halide or methosulfate; N,N-diplinoleioylethyl)-N,N-dipropylammonium salt of halide or methosulfate, and mixtures thereof.

A particularly preferred diester quat has the structure (II) below.

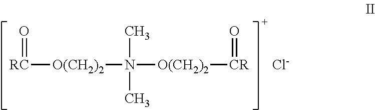

wherein R is a 15 carbon radical.

Useful commercially available diester quats are Varisoft® EQ 65 (distearoylethyl dimethylammonium chloride) and Rewoquat® WE 38, both available from Evonik Goldschmidt GmbH.

Amounts of the diester quat may range from about 0.05 to about 10%, preferably from 0.5 to 6%, more preferably from 1 to 4% and optimally from 2 to 3% by weight of the composition.

The friction enhancing agent of the present invention is a salt of structure AB, wherein A is a cationic charged component of the salt AB, and B is an anionic charged component of the salt AB, A has at least one quaternized nitrogen atom, at least two hydroxyl groups and a molecular weight no higher than about 400.

Cationic charged component A will have at least one, preferably from one to four, optimally one to two quaternized nitrogen atoms. Further, A will have at least two hydroxyl groups, preferably from two to three hydroxyl groups.

Anionic charged component B may be organic or inorganic with proviso that the material is cosmetically acceptable. Typical inorganic anions are halides, sulfates, phosphates, nitrates and borates. Most preferred are the halides, especially chloride. Organic anionic counter ions include methosulfate, toluoyl sulfate, acetate, citrate, tartrate, lactate, gluconate, and benzenesulfonate. The number and charge of negatively charged component B will be sufficient to neutralize the positive charge of component A.

A preferred embodiment of the friction enhancing agent are the dihydroxypropyl tri(C₁-C₃ alkyl) ammonium salts. These salts may be obtained in a variety of synthetic procedures, most particularly by hydrolysis of chlorohydroxypropyltri(C₁-C₃ alkyl)ammonium salts. Ordinarily the C₁-C₃ alkyl constituent on the quaternized ammonium group will be methyl, ethyl, n-propyl or isopropyl and mixtures thereof. Particularly preferred is a trimethyl ammonium group known through INCI nomenclature as a “trimonium” group. A most preferred species is 1,2-dihydroxypropyltrimonium chloride, wherein the C₁-C₃ alkyl is a methyl group.

Another preferred embodiment is the dihydroxypropyl di(C₁-C₃ alkyl) mono(hydroxyethyl) ammonium salts. Most preferred is the material of structure (III).

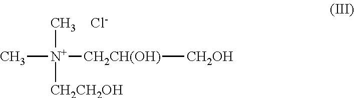

Amounts of the friction enhancing agent may range from about 0.05 to about 10%, preferably from about 0.1 to about 8%, more preferably from about 1 to about 6%, optimally from about 2 to about 5% by weight of the composition.

By the term personal care composition is meant any substance applied to a human body for improving appearance, cleansing, odor control or general aesthetics. Nonlimiting examples of personal care compositions include leave-on skin lotions and creams, shampoos, hair conditioners, shower gels, toilet bars, antiperspirants, deodorants, dental products, shave creams, depilatories, lipsticks, foundations, mascara, sunless tanners and sunscreen lotions.

Compositions of this invention will also include a cosmetically acceptable carrier. Amounts of the carrier may range from about 1 to about 98%, preferably from about 50 to about 95%, optimally from about 65 to about 90% by weight of the composition. Among the useful carriers are water, emollients, fatty acids, fatty alcohols, thickeners and combinations thereof. The carrier may be aqueous, anhydrous or an emulsion. Preferably the compositions are aqueous, especially water and oil emulsions of the W/O or O/W or triplex W/O/W variety.

Amounts of the water when present may range from 20 to 98%, preferably from 40 to 95%, more preferably from 60 to 90%, and optimally from 75 to 90% by weight of the composition.

Emollient materials when present may be in the form of natural or synthetic esters and hydrocarbons.

Among the ester emollients are:

a) Alkenyl or alkyl esters of fatty acids having 10 to 20 carbon atoms. Examples thereof include isopropyl palmitate, isoarachidyl neopentanoate, isononyl isonanonoate, oleyl myristate, isopropyl myristate, oleyl stearate, stearyl stearate, and oleyl oleate.

b) Ether-esters such as fatty acid esters of ethoxylated fatty alcohols.

c) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

d) Wax esters such as beeswax, spermaceti wax and tribehenin wax.

e) Sterols esters, of which cholesterol fatty acid esters are examples thereof.

f) Sugar esters of fatty acids such as sucrose polybehenate and sucrose polycottonseedate.

g) Natural esters useful in this invention are sunflower seed oil, safflower oil, cottonseed oil, olive oil, jojoba and mixtures thereof.

Hydrocarbons which are suitable emollients include petrolatum, mineral oil, C₁₁-C₁₃ isoparaffins, polyalphaolefins, and combinations thereof.

Fatty alcohols having from 10 to 30 carbon atoms are further useful carriers. Illustrative are stearyl alcohol, lauryl alcohol, myristyl alcohol and cetyl alcohol.

Amounts may range from 0.05 to 20%, preferably from 1 to 10% by weight of the composition.

Fatty acids having from 10 to 30 carbon atoms may in certain formulations also be suitable as carriers. Illustrative of this category are pelargonic, lauric, myristic, palmitic, stearic, isostearic, hydroxystearic, oleic, linoleic, ricinoleic, arachidic, behenic and erucic acids. Particularly preferred is stearic acid.

Thickeners can also be utilized as cosmetically acceptable carriers. Typical thickeners include polyacrylamides (e.g. Sepigel 305®), acryloyldimethyltaurate polymers and copolymers (e.g. Aristoflex AVC), crosslinked acrylates (e.g. Carbopol 982®), hydrophobically-modified acrylates (e.g. Carbopol 1382®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenum, pectin and combinations of these gums. Inorganics may also be utilized as thickeners, particularly clays such as bentonites and hectorites, fumed silicas, and silicates such as magnesium aluminum silicate (Veegum®). Also starches such as tapioca starch may be useful as thickeners.

Polyhydric alcohols may be employed in certain compositions of the present invention. Typical polyhydric alcohols include glycerin (also known as glycerol), polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of polyhydric alcohol when present may range from 0.1 to 40%, preferably from 0.5 to 20%, optimally from 1 to 10% by weight of the composition.

Polysiloxane materials may be present in compositions of this invention. The organopolysiloxane may be volatile, nonvolatile, or a mixture of volatile and non-volatile silicones. The term “nonvolatile” refers to those silicones that are liquid or solid under ambient conditions and have a flash point (under one atmosphere pressure) of at least about 100° C. The term “volatile” refers to all other silicone oils. Suitable organopolysiloxanes include polyalkylsiloxanes, cyclic polyalkylsiloxanes, and polyalkylarylsiloxanes.

Polyalkylsiloxanes can be represented by the general chemical formula R₃SiO[R₂SiO]ₓSiR₃ wherein R is an alkyl group having from one to about 30 carbon atoms (preferably R is methyl or ethyl) and x is an integer from 0 to about 10,000, chosen to achieve the desired molecular weight which can range to over about 10,000,000. Commercially available polyalkylsiloxanes include the polydimethylsiloxanes, which are also known as dimethicones. These include the Viscasil® series sold by the Momentive Chemical Company and the Dow Corning® 200 series sold by Dow Corning Corporation. Dimethicones include those represented by the chemical formula (CH₃)₃SiO[(CH₃)₂SiO]ₓ[CH₃RSiO]_{y}Si(CH₃)₃ wherein R is straight or branched chain alkyl having from 2 to about 30 carbon atoms and x and y are each integers of 1 or greater selected to achieve the desired molecular weight which can range to over about 10,000,000. Examples of these alkylsubstituted dimethicones include cetyl dimethicone and lauryl dimethicone.

Cyclic polyalkylsiloxanes suitable for use in the composition include those represented by the chemical formula [SiR₂—O]ₙ wherein R is an alkyl group (preferably R is methyl or ethyl) and n is an integer from about 3 to about 8, more preferably from 4 to 6. Where R is methyl, these materials are typically referred to as cyclomethicones. Commercially available cyclomethicones include Dow Corning® 244 fluid which primarily contains the cyclomethicone tetramer (i.e. n=4), Dow Corning® 344 fluid which primarily contains the cyclomethicone pentamer (i.e. n=5), Dow Corning® 245 which primarily contains a mixture of the cyclomethicone tetramer and pentamer (i.e. n=4 and 5), and Dow Corning® 345 which primarily contains a mixture of the cyclomethicone tetramer, pentamer, and hexamer (i.e. n=4, 5 and 6).

Also useful are materials such as trimethylsiloxysilicate, which is a polymeric material corresponding to the general chemical formula [(CH₂)₃SiO_{1/2}]ₓ[SiO₂]_{y}, wherein x is an integer from about 1 to about 500 and y is an integer from about 1 to about 500. A commercially available trimethylsiloxysilicate is sold as a mixture with dimethicone as Dow Corning® 593 fluid.

Dimethiconols are also suitable for use in the composition. These compounds can be represented by the chemical formulas R₃SiO[R₂SiO]_{X}SiR₂OH and HOR₂SiO[R₂SiO]_{X}SiR₂OH wherein R is an alkyl group (preferably R is methyl or ethyl) and x is an integer from 0 to about 500, chosen to achieve the desired molecular weight. Commercially available dimethiconols are typically sold as mixtures with dimethicone or cyclomethicone (e.g. Dow Corning® 1401, 1402, 1403 and 1501 fluids). Particularly preferred is a blend with INCI name of Cyclopentasiloxane and PEG/PPG-20/15 Dimethicone commercially available from Momentive as SF1528.

Crosslinked organopolysiloxane elastomers may also be useful as polysiloxane materials. These may be of the emulsifying or non-emulsifying crosslinked elastomer variety. The term “non-emulsifying” defines a crosslinked organopolysiloxane elastomer from which polyoxyalkylene units are absent. The term “emulsifying” is used to mean crosslinked organopolysiloxane elastomer having at least one polyoxyalkylene unit.

Non-emulsifying silicone elastomers may be powders such as vinyl dimethicone/methicone silesquioxane crosspolymers available from Shin-Etsu as KSP-100, KSP-101, KSP-102, KSP-103, KSP-104, KSP-105, hybrid silicone powders that contain a fluoroalkyl group such as KSP-200, and hybrid silicone powders that contain a phenyl group such as KSP-300; and Dow Corning material DC 9506.

Preferred organopolysiloxane compositions are dimethicone/vinyl dimethicone crosspolymers. These are commercially available as Dow Corning (DC 9040 and DC 9045), Momentive Chemical (SFE 839), Shin Etsu (KSG-15, 16, 18 [dimethicone/phenyl vinyl dimethicone crosspolymer]), and Grant Industries (Gransil™ line of materials), and lauryl dimethicone/vinyl dimethicone crosspolymers supplied by Shin Etsu as KSG-31, KSG-32, KSG-41, KSG-42, KSG-43, and KSG-44.

Amounts of the polysiloxane materials may range from 0.1 to 80%, preferably from 1 to 60%, optimally from 5 to 40% by weight of the composition.

Nonionic emulsifiers or surfactants may also be present in compositions of the present invention. Total concentration of the emulsifier when present may range from 0.01 to 15%, preferably from 0.1 to 5%, optimally from 1 to 3% by weight of the composition. Particularly preferred nonionic emulsifiers are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) are also suitable nonionic emulsifiers. Compositions of this invention may also contain anionic, cationic and/or amphoteric emulsifiers or surfactants.

Sunscreen actives may also be included in the compositions. Particularly preferred are such materials as ethylhexyl p-methoxycinnamate, available as Parsol MCX®, Avobenzene, available as Parsol 17890 and benzophenone-3, also known as Oxybenzone. Inorganic sunscreen actives may be employed such as microfine titanium dioxide and zinc oxide. Amounts of the sunscreen agents when present may generally range from 0.1 to 30%, preferably from 2 to 20%, optimally from 4 to 10% by weight.

Compositions of the present invention may also contain vitamins. Illustrative water-soluble vitamins are Niacinamide, Vitamin B₂, Vitamin B₆, Vitamin C, Folic Acid and Biotin. Among the useful water-insoluble vitamins are Vitamin A (retinol), Vitamin A Palmitate, Ascorbyl Tetraisopalmitate, Vitamin E (tocopherol), Vitamin E Acetate and DL-panthenol. Total amount of vitamins when present in compositions according to the present invention may range from 0.0001 to 10%, preferably from 0.01% to 1%, optimally from 0.1 to 0.5% by weight of the composition.

Desquamation agents are further optional components. Illustrative are the alpha-hydroxycarboxylic acids and beta-hydroxycarboxylic acids. Among the former are salts of glycolic acid, lactic acid and malic acid. Salicylic acid is representative of the beta-hydroxycarboxylic acids. Amounts of these materials when present may range from 0.01 to 15% by weight of the composition.

A variety of herbal extracts may optionally be included in compositions of this invention. Illustrative are green tea, chamomile, licorice, lavender, grape seed and extract combinations thereof. The extracts may either be water soluble or water-insoluble carried in a solvent which respectively is hydrophilic or hydrophobic. Water and ethanol are the preferred extract solvents.

Colorants, fragrances, opacifiers and abrasives may also be included in compositions of the present invention. Each of these substances may range from 0.05 to 5%, preferably each between 0.1 and 3% by weight.

Preservatives can desirably be incorporated into the cosmetic compositions of this invention to protect against the growth of potentially harmful microorganisms. Suitable traditional preservatives for compositions of this invention are alkyl esters of para-hydroxybenzoic acid. Other preservatives which have more recently come into use include hydantoin derivatives, propionate salts, and a variety of quaternary ammonium compounds. Particularly preferred preservatives are phenoxyethanol, ethylenediaminetetraacetic acid salts (EDTA), methyl paraben, propyl paraben, DMDM Hydantoin, iodopropynyl butylcarbamate, 5-chloro-2-methyl-4-isothiazolin-3-one, 2-methyl-4-isothiazolin-3-one and benzyl alcohol. The preservatives should be selected having regard for the use of the composition and possible incompatibilities between the preservatives and other ingredients in the emulsion. Preservatives are preferably employed in amounts ranging from 0.00001% to 2% by weight of the composition.

The term “comprising” is meant not to be limiting to any subsequently stated elements but rather to encompass non-specified elements of major or minor functional importance. In other words the listed steps, elements or options need not be exhaustive. Whenever the words “including” or “having” are used, these terms are meant to be equivalent to “comprising” as defined above.

Except in the operating and comparative examples, or where otherwise explicitly indicated, all numbers in this description indicating amounts of material ought to be understood as modified by the word “about”.

The following examples will more fully illustrate the embodiments of this invention. All parts, percentages and proportions referred to herein and in the appended claims are by weight unless otherwise illustrated.

### Example 1

A series of typical body lotions were formulated and are detailed in Table I.

TABLE I

| | Sample (Weight %) | | | | |
|---|---|---|---|---|---|
| Ingredient | A | B | C | D | E |
| Water, deionized | 83.965 | 83.965 | 84.965 | 84.865 | 79.965 |
| Methylparaben | 0.200 | 0.200 | 0.200 | 0.200 | 0.200 |
| Disodium EDTA | 0.050 | 0.050 | 0.050 | 0.050 | 0.050 |
| Sodium Chloride | 0.100 | 0.100 | 0.100 | 0.100 | 0.100 |
| Distearyldimonium | 2.750 | — | — | — | — |
| Chloride | | | | | |
| Dipalmitoylethyl | — | 2.750 | 2.750 | 2.750 | 2.750 |
| dimonium chloride | | | | | |
| Cetyl Alcohol | 4.710 | 4.710 | 4.710 | 4.710 | 4.710 |
| Steareth-21 | 1.725 | 1.725 | 1.725 | 1.725 | 1.725 |
| Petrolatum G2212 | 0.250 | 0.250 | 0.250 | 0.250 | 0.250 |
| Isopropyl Palmitate | 4.000 | 4.000 | 4.000 | 4.000 | 4.000 |
| Mineral Oil 1000 sus | 0.750 | 0.750 | 0.750 | 0.750 | 0.750 |
| Propylparaben | 0.100 | 0.100 | 0.100 | 0.100 | 0.100 |
| Phenoxyethanol | 0.400 | 0.400 | 0.400 | 0.400 | 0.400 |
| Dihydroxypropyltri- | 1.000 | 1.000 | — | 0.100 | 5.000 |
| monium Chloride | | | | | |
| (“Glycerol Quat”) | | | | | |

The Samples of Table I were evaluated for their relative friction properties. This value was utilized to identify relative ability to impart a draggy effect onto skin.

### Procedure

The test is conducted in an environmentally controlled chamber at 21° C. and 30% relative humidity. Sample size of 0.1 mL is spread on a glass plate over a 6 by 2 inch area (2.54 cm to the inch). The plate is attached to the cross-head of an Instron Model 4501 Materials Testing System. A 3 by 1 inch aluminum sled is fitted with a rectangular foam pad 3 inches by 1 inch by 0.125 inch (Foamex® L2185040B2). A 100 gram weight is placed on the sled. It is then pulled across the same area at a rate of 10 cm/min., and a friction measurement taken immediately thereafter. The integral of force vs. a distance of 40 mm (i.e.—amount of work of dynamic slip friction units of gram-mm) is calculated for each of the intervals. Also measured is the static load (grams) or stick. Three runs are conducted for each product. Results are recorded in Table II.

TABLE II

| | Coefficient of Static Friction | Dynamic Friction or Work |
|---|---|---|
| Sample | (Force in Grams at 0 min.) | (Gram-mm at 0 min.) |
| A | 8.00 | 400 |
| B | 10.80 | 498 |
| C | 9.90 | 420 |
| D | 10.20 | 540 |
| E | 11.20 | 595 |

Sample A and B evaluate the effect of 1% glycerol quat on lotions comparing distearyldimonium chloride (“dialkyl quat”) to diester quat. The higher values for Sample B indicates that in a diester quat system greater dragginess is imparted by the glycerol quat. Sample C is the formula wherein the glycerol quat is absent. Both the coefficient of static friction and Work are lower values than in Sample B. The lower values demonstrate dragginess for the combined diester quat and glycerol quat. As the amount of glycerol quat increases from 0.1% to 5%, dragginess increases. Compare Sample E to D.

What is claimed is:

1. A personal care composition comprising:
(i) from about 0.05 to about 10% by weight of a diester quat of the following structure (I): 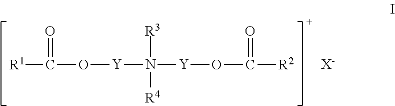
wherein R¹ and R² are independently selected from branched or unbranched, saturated or unsaturated radicals having from 13 to 23 carbon atoms; R³ and R⁴ are independently selected from an alkyl radical having from 1 to 4 carbon atoms; Y is independently selected from saturated or unsaturated alkylene radicals having from 2 to 8 carbon atoms; and X is an organic or inorganic anion;
(ii) from about 0.05% to about 10% by weight of a friction enhancing agent which is a salt of structure AB,
wherein A is a cationic charged component of the salt AB,
B is an anionic charged component of the salt AB, and
A has at least one quaternized nitrogen atom, at least two hydroxyl groups and a molecular weight no higher than about 400; and
(iii) from about 1% to about 98% by weight of a cosmetically acceptable carrier.

2. The composition according to claim 1 wherein the diester quat has the structure II: 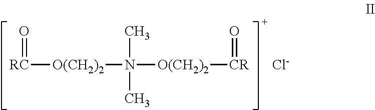
wherein R is a 15 carbon radical.

3. The composition according to claim 1 wherein the diester quat is a N,N-di(β-palmitoylethyl)-N,N-dimethylammonium salt.

4. The composition according to claim 1 wherein the diester quat is present in an amount from 1 to 4% by weight of the composition.

5. The composition according to claim 1 wherein the X anion is selected from the group consisting of halide, nitrate, acetate, sulfate, phosphate, tosylate, methosulfate and combinations thereof.

6. The composition according to claim 1 wherein the X anion is chloride.

7. The composition according to claim 1 wherein the friction enhancing agent is a dihydroxypropyl tri(C₁-C₃ alkyl) ammonium salt.

8. The composition according to claim 1 wherein the friction enhancing agent is dihydroxypropyltrimonium chloride.

9. The composition according to claim 1 wherein the friction enhancing agent is present in an amount from about 0.1 to about 8% by weight of the composition.
